(19) United States
(12) Patent Application Publication
(10) Pub. No.: US 2009/0013997 A1
(43) Pub. Date: Jan. 15, 2009

(54) INHALANT EXPOSURE SYSTEM

(76) Inventors: Roy Edmund Barnewall, Columbus, OH (US); Richard Scott Tuttle, Overland Park, KS (US)

Correspondence Address: BATTELLE MEMORIAL INSTITUTE, 505 KING AVENUE, COLUMBUS, OH, 43201-2693, US

(21) Appl. No.: 12/088,454

(22) PCT Filed: Sep. 29, 2006
(86) PCT No.: PCT/US2006/038130

§ 371 Date: Aug. 1, 2008

Related U.S. Application Data
(60) Provisional application No. 60/722,838, filed on Sep. 30, 2005.

(51) Int. Cl. A61M 16/00; A01K 1/03

(52) U.S. Cl. 128/203.15; 119/420

(57) ABSTRACT

An inhalant exposure unit and system that provides controlled flow of inhalant to an animal with a breathing system that provide controlled exposure of inhalant, minimized breathing of exhaled air and control of exhaust flow.

### FIELD OF THE INVENTION

The invention provides a method and apparatus for controlled testing of single and multiple animals with selected inhalants. The invention provides for reduced rebreathing of exhaled breath.

### BACKGROUND OF THE INVENTION

Various inhalation exposure apparatus have been developed for providing controlled levels of inhalants to animals with the purpose of determining the impact on the animals. One of the primary considerations for inhalation exposure systems is that the inhaled materials be of the same concentration so that biological effects observed on the teat animals can be correlated and reproducibly obtained.

Recent world events have lead to increased concern of potential terrorist biological warfare attacks. One of the main biological warfare threats to humans is inhalational exposure to pathogenic bioaerosols. Examples of infectious diseases known to be caused by aerosolized bacteria are tuberculosis, legionellosis, and anthrax. Bacteria are single celled organisms with sizes from 0.3 to 10 um. Anthrax, a serious illness caused by the bacterium Bacillus anthracis, is considered one of the prototypical biological warfare biological warfare agents. One of the greatest bioaerosol threats is the inhalational exposure to Bacillus anthracis. The spore-forming ability makes Bacillus anthracis well suited for multiple delivery methods, which include liquid or dry agent disseminations. To protect against biological warfare attacks, various vaccines and post-exposure treatment approaches must be evaluated. To fully evaluate the efficacy of vaccines and therapeutics against bioaerosols of biological warfare agents, a well characterized and reproducible inhalant exposure system is needed. The inhalant exposure system and inhalant procedures, as much as possible, should follow Good Laboratory Practice regulations to support such studies for licensing of these products. The present invention includes the design, construction and initial characterization of an inhalation exposure system that can be used to challenge single to multiple animal models to support animal inhalation exposure testing of various products.

### BRIEF DESCRIPTION OF THE INVENTION

A first broad embodiment of the invention includes an n inhalant exposure unit having a housing positioned around a central axis having an inlet end and an outlet end. A face plate is positioned vertically to the central axis at the outlet end of the housing but not in contact therewith. An annular outlet is formed by the spaced apart relationship of the outlet end and the face plate. The face plate has an axial opening for admitting at least a portion of an animal's head. In one embodiment the annular outlet is typically totally unimpeded by supports and the like so as to not impede the flow of inhalant and exhaled breath. In some embodiments, however, there may be one to several struts or supports such as those typically used in the art, that do not substantially interfere with the flow of inhalant and exhaled breath.

A yet further embodiment provides for an inhalant exposure unit having a housing 201 positioned around a central axis having an inlet end and an outlet end. At least a portion of the housing for this embodiment forms a truncated cone. The sides of the truncated cone form an angle θ with respect to the central axis. Typically the angle θ has a value of about 0° to about 60°. A face plate is positioned vertically to the central axis 103 at the outlet end of the housing but not in contact therewith. An annular outlet is formed by the spaced apart relationship of the outlet end of the truncated cone and the face plate. The face plate has an axial opening for admitting at least a portion of an animal's head. The benefits of the invention are obtained by having the flow of inhalant flow past the nostrils and/or mouth of the animal and sweep exhaled breath away from the animal's nose or mouth and into the annular outlet. The annular outlet is typically unimpeded by supports and the like so as to not impede the flow of inhalant and exhaled breath. In some embodiments, however, there may be one to several struts or supports such as those typically used in the art, that do not substantially interfere with the flow of inhalant and exhaled breath through the annular outlet. The conical shape the housing provides for enhanced flow of inhalant past the animal's head compared to the first embodiment that does not use a truncated cone. Both embodiments, however, provide for substantially unimpeded flow of inhalant in a 360° pattern around the animal's head so as to sweep exhaled air away from the animal's nose and mouth.

A yet further embodiment of the invention provides for an inhalant exposure unit having a housing 301 positioned around a central axis having an inlet end and an outlet end. The housing typically forms at least in part a truncated cone. The sides of the truncated cone form an angle θ with respect to the central axis. Typically the angle θ has a value of about 0° to about 60°. A face plate is positioned vertical to the central axis at the outlet end of the housing but not in contact therewith. An annular outlet is formed by the spaced apart relationship of the outlet end and face plate. An outer housing located concentrically around the axis and housing. The outer housing and the housing together form an exhaust passage between them. The outer housing has a back end that corresponds to the inlet end of housing and a front end that aligns with the outlet end of the housing. The front end of the outer housing, however, makes contact with the face plate in a sealing relationship to prevent the loss of inhalant and exhaled breath. The face plate has an axial opening for admitting at least a portion of an animal's head. Typically the animal's head is admitted through the axial opening into the exposure volume around the animals head. The benefits of the invention are obtained by having the flow of inhalant flow past the nostrils and/or mouth of the animal and sweep exhaled breath away from the animal's nostrils or mouth and into the annular outlet. The annular outlet is typically unimpeded by supports and the like so as to not impede the flow of inhalant and exhaled breath. In some embodiments, however, there may be one to several struts or supports (not shown) such as those typically used in the art, that do not substantially interfere with the flow of inhalant and exhaled breath through the annular outlet. The conical shape of housing provides for enhanced flow of inhalant past the animal's head compared to the embodiment that does not use a truncated cone. All embodiments, however, provide for substantially unimpeded flow of inhalant in a 360° pattern around the animal's head so as to sweep exhaled air away from the animal's nostrils and/or mouth. The inhalant and exhaled breath flow into annular outlet and then through the exhaust passage to an outlet. A flow restrictor may be used to further control the flow of inhalant and exhaled breath to the exhaust outlet.

A yet further embodiment of the invention includes an inhalation exposure system for treatment of a patient including an inhalation generator for providing an aerosol or powder; and an inhalation exposure unit having an inlet connected to the inhalation generator that includes

1. a tapered exposure chamber having its inlet at a narrow end and having a port at the wide end of the chamber that accommodates at least a part of a patient's head for breathing from the exposure chamber;

2. an exhaust passage for air flow having an inlet connected to the wider portion of the tapered exposure chamber, and having an outlet, and

3. a flow restrictor in the exhaust passage; and a vacuum unit that provides a vacuum at the outlet of the exhaust passage. Typically the inhalation generator is a nebulizer. The patient to be treated is typically a human or animal.

### BRIEF DESCRIPTION OF THE DRAWINGS

FIG. 1 is a schematic of one embodiment of an aerosol exposure system.

FIG. 2 is a schematic of another embodiment of an aerosol exposure system.

FIG. 3 is a schematic of a yet further embodiment of an aerosol exposure system.

FIG. 4 is a schematic of a yet additional embodiment of an aerosol exposure system.

FIG. 5 is a schematic of one embodiment of an inhalant exposure system for single exposures.

FIG. 6 is a bar graph with sample time (seconds) on the horizontal scale and APS particle sizer counts at 1:1 dilution on the vertical scale. The data shows new aerosol system stability of aerosolized B. globigii spore counts over time. The tests were performed at 30 PSI.

FIG. 7 is a bar graph depicting sample time (seconds) on the horizontal scale and APS particle sizer counts at 1:1 dilution on the vertical scale. The data shows current aerosol system stability of aerosolized B. globigii spore counts over time. Collison apparatus pressure is at 30 psi.

FIG. 8 is a graph depicting an aerosol size distribution plot for aerosolized B. anthracis (triangles) and B. globigii (circles). Spore size (um) is plotted on the horizontal scale and percent mass is plotted on the vertical scale.

FIG. 9 is a schematic of another embodiment of the inhalant exposure system for dual exposures.

FIG. 10 is a bar graph of a particle sizer correlation.

FIG. 11 is a graph depicting PSL system homogeneity and aerosol delivery efficiency results for 0.993 um particles.

FIG. 12 is a graph depicting PSL system homogeneity and aerosol delivery efficiency results for 1.992 um particles. The horizontal scale is Time in seconds and the vertical scale is the number of particle counts.

FIG. 13 is a graph depicting PSL system homogeneity and aerosol delivery efficiency results for 2.92 um particles. The horizontal scale is Time in seconds and the vertical scale is the number of particle counts.

FIG. 14 is a bar graph of B. anthracis aerosol delivery efficiency. The horizontal scale is Time in seconds.

FIG. 15 is a graph of B. anthracis aerosol stability. The horizontal scale shows Time in seconds and the vertical scale shows Raw Particle Counts.

### DETAILED DESCRIPTION OF THE INVENTION AND BEST MODE

Broadly the invention provides for an inhalant exposure system for animals that improves the exposure for the animal. The unit provides for low volume displacement providing fast aerosol stabilization and washout. Typically the unit allows near isokinetic sampling that allows the collection of a truer aerosol sample representative of the exposure concentration. The system typically has a flow over muzzle design with exhaust located around the periphery of the animal's neck or head so as to reduce or eliminate aerosol and exhaled air rebreathing. Pressure fluctuation effects and rebreathing of exhaled air on aerosol deliver are also minimized by a vacuum at the exhaust port and a flow restrictor in the exhaust passage. Additionally, the concentric exhaust system provides for more uniform distribution of aerosol in the animal breathing zone prior to exhaust treatment.

The dual unit or multiple unit typically has the ability to expose each animal at different durations based on respiration rate. Typically, each unit has isolation gate valves with fresh air delivery independently for each exposure location. In some embodiments, use of a single sampler for concentration analysis for exposure dose measurement eliminates multiple sample analysis. In other embodiments, pressure and vacuum respiration relief dampers reduce animal respiration effects on aerosol and system flow dynamics and control.

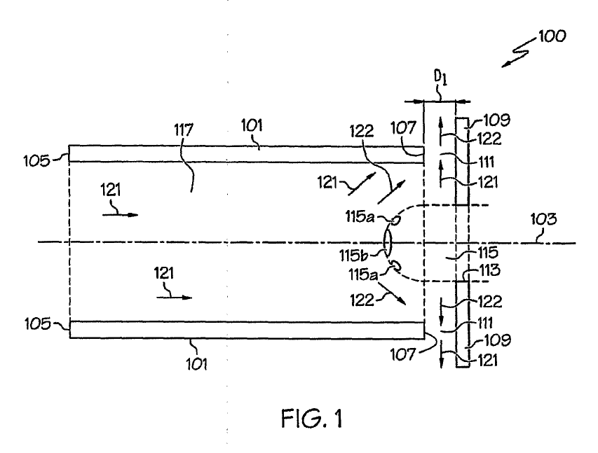

Referring now to FIG. 1, this figure shows a schematic of one embodiment of the invention for an inhalant exposure unit 100. A housing 101 is positioned around a central axis 103 having an inlet end 105 and an outlet end 107. A face plate 109 is positioned vertically to the central axis 103 at the outlet end 105 of the housing 101 but not in contact therewith. An annular outlet 111 is formed by the spaced apart relationship of the outlet end 107 and the face plate 109. The face plate has an axial opening 113 for admitting at least a portion of an animal's head 115. Typically the animal's head 115 is admitted through the axial opening 113 into the exposure volume 117. The animal is typically positioned and the size of the axial opening 113 adjusted so that the animal's breathing openings, such as the nostrils and/or mouth, extend into the exposure volume 117 to at least the outlet end 107 of housing 101. Most preferably, to fully realized the benefits of the present invention, the nostrils and/or mouth should extend beyond the outlet end 107 of housing 101. If a nose only or mouth only breathing is used, these considerations only apply to the respective breathing opening. The benefits of the invention are obtained by having the flow of inhalant 121 flow past the nostrils and/or mouth of the animal and sweep the exhaled breath away from the animal's nose or mouth and into the annular outlet 111. The annular outlet 111 is typically totally unimpeded by supports and the like so as to not impede the flow of inhalant and exhaled breath. In some embodiments, however, there may be one to several struts or supports (not shown) such as those typically used in the art, that do not substantially interfere with the flow of inhalant and exhaled breath.

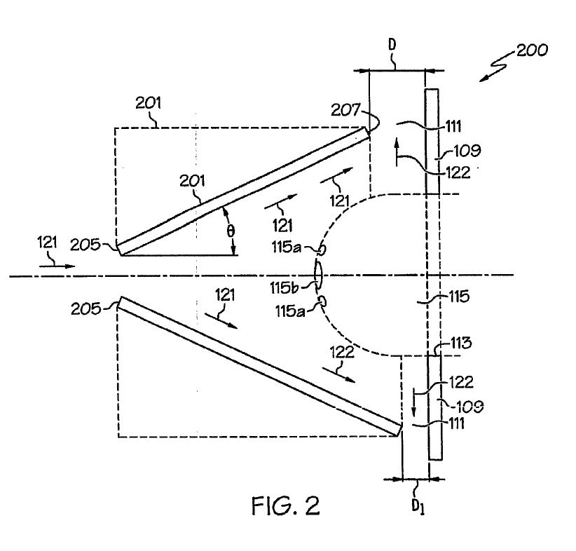

Referring now to FIG. 2, this figure shows a schematic of another embodiment of the invention for an inhalant exposure unit 200. A housing 201 is positioned around a central axis 103 having an inlet end 205 and an outlet end 207. The housing 201 in this embodiment forms a truncated cone. The sides of the truncate cone form an angle θ with respect to the central axis. Typically the angle θ has a value of about 0° (the first embodiment above) to about 60°. The angle chosen dependent on the size and facial configurations of animal to be exposed to the inhalant. A face plate 109 is positioned vertically to the central axis 103 at the outlet end 205 of the housing 201 but not in contact therewith. An annular outlet 111 is formed by the spaced apart relationship of the outlet end 207 and the face plate 109. The face plate has an axial opening 113 for admitting at least a portion of an animal's head 115. Typically the animal's head 115 is admitted through the axial opening 113 into the exposure volume 217. The animal is typically positioned and the size of the axial opening 113 adjusted so that the animal's breathing openings, such as the nostrils and/or mouth, extend into the exposure volume 217 to at least the outlet end 207 of housing 201. Most preferably, to fully realize the benefits of the present invention, the nostrils and/or mouth should extend beyond the outlet end 207 of housing 201. If a nose only or mouth only breathing is used, these considerations only apply to the respective breathing opening. The benefits of the invention are obtained by having the flow of inhalant 121 flow past the nostrils and/or mouth of the animal and sweep exhaled breath away from the animal's nose or mouth and into the annular outlet 111. The annular outlet 111 is typically unimpeded by supports and the like so as to not impede the flow of inhalant and exhaled breath. In some embodiments, however, there may be one to several struts or supports (not shown) such as those typically used in the art, that do not substantially interfere with the flow of inhalant and exhaled breath through the annular outlet 111.

The conical shape the housing 210 provides for enhanced flow of inhalant 121 past the animal compared to the first embodiment that does not use a truncated cone. Both embodiments, however, provide for substantially unimpeded flow of inhalant 121 in a 360° pattern around the animal's head so as to sweep exhaled air away from the animal's nose and mouth.

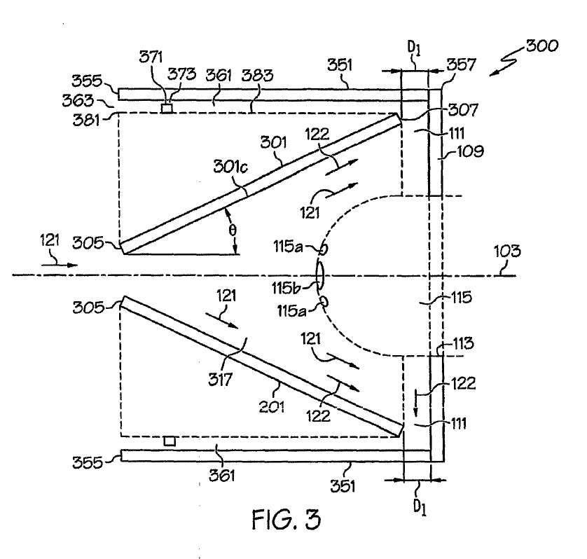

Referring now to FIG. 3, the figure shows a schematic of a yet further embodiment of the invention for an inhalant exposure unit 300. A housing 301 is positioned around a central axis 103 having an inlet end 305 and an outlet end 307. The housing 301 typically forms a truncated cone 301c. The sides of the truncated cone 301c form an angle θ with respect to the central axis 103. Typically the angle θ has a value of about 0° (the first embodiment above) to about 60°. The angle chosen dependent on the size and facial configurations of animal to be exposed to the inhalant. A face plate 109 is positioned vertical to the central axis 103 at the outlet end 305 of the housing 301 but not in contact therewith. An annular outlet 111 is formed by the spaced apart relationship of the outlet end 307 and face plate 109. An outer housing 351 is located concentrically around axis 103 and housing 301. Outer housing 351 and housing 301 together form an exhaust passage 361 between them. The outer housing 351 has a back end 355 that corresponds to the inlet end 305 of housing 301, and a front end 357 that aligns with the outlet end 307 of the housing 301. The front end of outer housing 351, however, makes contact with face plate 109 in a sealing relationship to prevent the loss of inhalant and exhaled breath 122.

The face plate has an axial opening 113 for admitting at least a portion of an animal's head 115. Typically the animal's head 115 is admitted through the axial opening 113 into the exposure volume 317. The animal and the animal's head 115 is typically positioned and the size of the axial opening 113 adjusted so that the animal's breathing openings, such as the nostrils 115a and/or mouth 115b, extend into the exposure volume 317 to at least the outlet end 307 of housing 301. Most preferably, to fully realize the benefits of the present invention, the nostrils 115a and/or mouth 115b should extend beyond the outlet end 307 of housing 301 into the treating volume 317. If nose only or mouth only breathing is used, these considerations only apply to the respective breathing opening. The benefits of the invention are obtained by having the flow of inhalant 121 flow past the nostrils 115a and/or mouth 115b of the animal and sweep exhaled breath away from the animal's nostrils or mouth and into the annular outlet 111. The annular outlet 111 is typically unimpeded by supports and the like so as to not impede the flow of inhalant 121 and exhaled breath 122. In some embodiments, however, there may be one to several struts or supports (not shown) such as those typically used in the art, that do not substantially interfere with the flow of inhalant and exhaled breath through the annular outlet 311.

The conical shape of housing 301 provides for enhanced flow of inhalant 121 past the animal's head 115 compared to the first embodiment that does not use a truncated cone. Both embodiments, however, provide for substantially unimpeded flow of inhalant 121 in a 360° pattern around the animal's head so as to sweep exhaled air away from the animal's nostrils 115a and/or mouth 115b. The inhalant 121 and exhaled breath 122 flow into annular outlet 311 and then through the exhaust passage 361 to an outlet 363. A flow restrictor 371 may be used to further control the flow of inhalant 121 and exhaled breath 122 to outlet 363.

In some embodiments, the housing 301 can be shaped as shown by dashed lines 381 to form a unitary structure having one surface 383 substantially parallel to outer housing 351 or any form in between. Flow restrictor 371 typically provides for an opening 373 between the flow restrictor 371 and outer housing 351. This flow restriction provides for more controlled flow of gases in that it is more difficult for the animal's breathing to reverse the flow of gases out of the unit. In some embodiments the flow restrictor 371 may completely close the space between the housing 301 and outer housing 351 and have a plurality of holes (not shown) in the flow to provide controlled flow of inhalant 121 and exhaled breath 122 out of the exhaust passage.

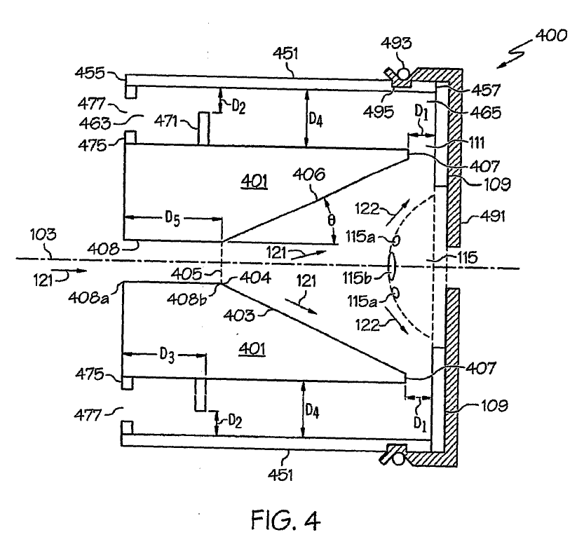

Referring now to FIG. 4, this figure is a schematic drawing depicting an additional embodiment of an inhalant exposure unit 400. Inhalant exposure unit 400 includes: A housing 401 located concentrically around a central axis 103 to form a partially a truncated cone 403 having a front end 407 and a back end 405, wherein the truncated cone 403 is located concentrically about axis 103 and having an inlet 404 at its narrow back end 405 and wherein the inner surface 406 of the truncated cone forms an angle θ with respect to the central axis 103; and an optional inlet tube 408 of length D₅ may be located concentrically within the housing 401 having an inlet 408a and an outlet 408b, the outlet 408b of the optional tube 408 operationally connected to the inlet 404 of the truncated cone 403; and an outer housing 451 around the housing 401 located concentrically around the central axis (typically forming an outer substantially tubular structure having a front 457 and back end 455 corresponding to that for the housing 401, and wherein the housing 401 and the outer housing 451 form an exhaust passage 461 between them having an exhaust 463 at the back of the exhaust passage 461 and an inlet 465 for the inhalant and exhaled breath; the exhaust 463 of the exhaust passage 461 is typically sealed in part by a back plate 475 that has one or more exhaust ports 477; a face plate 109 is placed vertical to the central axis 103 at the outlet end 407 of the housing 301 but not in contact therewith. An annular outlet 111 is formed by the spaced apart relationship of the outlet end 407 and face plate 109. An outer housing 451 is located concentrically around axis 103 and housing 401. Outer housing 451 and housing 401 together form an exhaust passage 461 between them. The outer housing 451 has a back end 455 that corresponds to the inlet end 405 of housing 401, and a front end 457 that aligns with the outlet end 407 of the housing 401. The front end of outer housing 451, however, makes contact with face plate 109 in a sealing relationship to prevent the loss of inhalant and exhaled breath 122.

The face plate has an axial opening 113 for admitting at least a portion of an animal's head 115. Typically the animal's head 115 is admitted through the axial opening 113 into the exposure volume 417. The animal and the animal's head 115 is typically positioned and the size of the axial opening 113 adjusted so that the animal's breathing openings, such as the nostrils 115a and/or mouth 115b, extend into the exposure volume 417 to at least the outlet end 407 of housing 401. Most preferably, to fully realize the benefits of the present invention, the nostrils 115a and/or mouth 115b should extend beyond the outlet end 407 of housing 401 into the treating volume 417. If nose only or mouth only breathing is used, these considerations only apply to the respective breathing opening. The benefits of the invention are obtained by having the flow of inhalant 121 flow past the nostrils 115a and/or mouth 115b of the animal and sweep exhaled breath away from the animal's nostrils or mouth and into the annular outlet 111, having an offset distance D₁. The annular outlet 111 is typically unimpeded by supports and the like so as to not impede the flow of inhalant 121 and exhaled breath 122. In some embodiments, however, there may be one to several struts or supports (not shown) such as those typically used in the art, that do not substantially interfere with the flow of inhalant and exhaled breath through the annular outlet 411.

The conical shape of housing 401 provides for enhanced flow of inhalant 121 past the animal's head 115 compared to the first embodiment that does not use a truncated cone. Both embodiments, however, provide for substantially unimpeded flow of inhalant 121 in a 360° pattern around the animal's head so as to sweep exhaled air away from the animal's nostrils 115a and/or mouth 115b. The inhalant 121 and exhaled breath 122 flow into annular outlet 411 and then through the exhaust passage 461 to an outlet 463. A flow restrictor 471 may be used to further control the flow of inhalant 121 and exhaled breath 122 to outlet 463. The flow restrictor 471 is typically located D₃ units from the exhaust end of the exhaust passage 461. Flow restrictor 471 forms an aperture D₂ in the exhaust passage 461. The aperture D₂ controls the flow rate of air as further discussed elsewhere herein. Exhaust passage 461 is typically concentric and has a sufficient volume to help damp the pulsating flow of gases produced due to the animal's breathing.

The following examples illustrate various embodiments of the invention. The examples are illustrative only and are not intended to limit the scope of the invention in any way.

For aerosol tests the following biological organisms were used. B. anthracis spores, Ames strain Lot B13, were produced from a single “parent” stock in 1% phenol and sterile water. “Parent” stocks were maintained at temperatures ranging from about 2 to about 8° C. Production was performed according to SOP MREF. X-098 “Production of Bacillus anthracis (hereafter B. anthracis) Spores.

A simulant used was: Polystyrene latex microspheres at sizes of 0.993, 1.992, and 2.92 um from Duke Scientific corp. The simulant is prepared as a suspension in deionized (DI) H₂O and reagent grade ethanol.

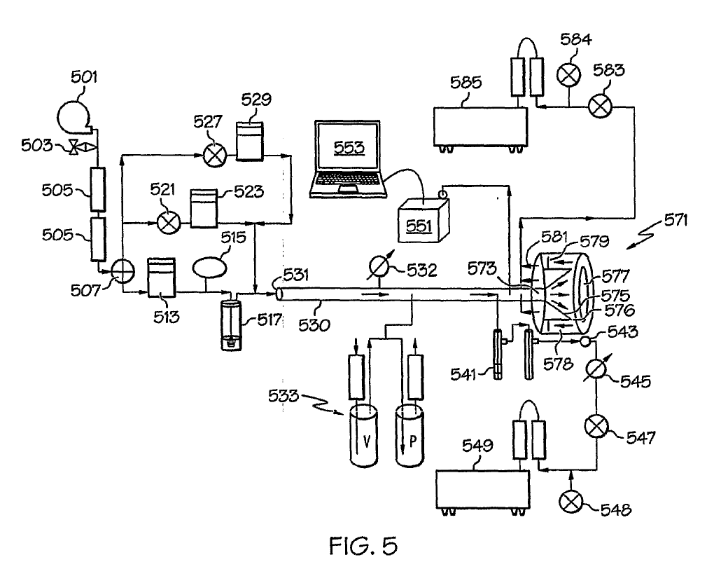

Referring now to FIG. 5, this figure illustrates a typical inhalant exposure unit for one animal with optional sensing instruments. A flow of air from a source 501 is controlled by pressure regulator 503 before flowing to one or more filters 505 (e.g. HEPA filters). A three way valve controls flow directly to a nebulizer 517 via a mass flow controller 513 and pressure gauge 515. A flow of dilution air flows via a pressure regulator 521 to mass flow controller 523 directly to the inlet 531 of tube 530. An additional flow path for bypass air involves flow from valve 507 to pressure regulator 527 and mass flow meter 529 and then directly to the tube inlet 531. Aerosol produced in the nebulizer 517 flows directly to the inlet 531 of tube 530. The length of tube 530 is any that provides a good flow aerosol flow path, distribution to sensing instruments and proper delivery to the inlet 573 of inhalant exposure unit 571. A differential pressure gauge 532 is typically used to monitor pressure in tube 530. Vacuum and pressure relief vessels 533 along with associated filters 534 (e.g. HEPA filters) may be used to control pressure in the tube. A sample collector such as an impinger 541 may connected to the tube 530 to collect aerosol particles. A critical orifice 543, along with vacuum gauge 545, valve 548 and a vacuum pump 549 along with filters 549A may be used to aid in collecting the samples. In one embodiment the critical orifice provides a flow of air of 2 L/min. An optional aerodynamic particle sizer 552 connected to the tube 530 may be used with a computer 553 to aid in monitoring and controlling particle size.

Aerosol and air then flows to the inlet 573 of the inhalant exposure unit 571 from which the flow is directed to a cone 575 where an animal's mouth and nose are typically placed via port 577. The unused aerosol and air along with exhaled air from the animal flows out of the cone 575 into a concentric inlet 576 to a typically concentric exhaust passage 578. Exhaust passage 578 contains a flow restrictor 579 that controls flow out of the exhaust passage and provides for increased air flow where the animal breathes in the cone 575. This is accomplished by a vacuum applied at an outlet port 581 of the exhaust passage 578. Flow restrictor 579 essentially controls the effects of this applied vacuum in the cone 575. As mentioned earlier the effect of the vacuum and flow restrictor 579 is to increase the speed of air flow at the animal's nose or mouth above the air flow provided by the inflow of air and aerosol to the cone. This has the effect of reducing rebreathing of exhaled air by the animal. Exhaust air flows from outlet port 581 to a valve 583 an optional bypass valve 584 and then to an exhaust pump 585 (with filters 585A) that provides vacuum at the outlet ports 581.

### Example 1

A laboratory scale inhalant exposure system for providing an inhalant such as an aerosol to animals was built in accordance with the figures. The inhalant exposure system was constructed of Plexiglas™ (although any plastic or metal inert to the test materials will work) and consisted of a 2.54 cm inside diameter tube with a 5.08 cm outside diameter of approximately 56 cm long. The end of the tube was mated with a 10.2 cm long and 5.1 cm inside diameter solid stock of Plexiglas™ with a 10.2 cm outside diameter. The end of the tube was lathed at 30° to form a truncated cone radiating out from the 2.54 cm (1 inch) diameter inner tube, to the 10.2 cm (4 inch) diameter outer tube for the insertion of the animals nose through a rubber dam. A 15.25 cm (6 inch) outside diameter and 12.7 cm (5 inch) inside diameter tube was mounted concentrically with front and back plates around the 10.2 cm (4 inch) diameter tube for exhausting the aerosol from the system. The face plate, located around the animal's nose insertion region, encompassed the cone and was spaced from the cone outlet end by an annular outlet gap of about 1.3 mm. The exhaust outlet increased the acceleration of resident aerosol that passed the animal's nose and/or mouth to facilitate the replenishment of fresh—low residence time biological aerosol in the animal's respiration zone. The aerosol then entered the exhaust passage which contained a flow restrictor which was separated about 3 mm from the outer housing. The flow restrictor acted as an exhaust flow distributor to maintain a consistent exhaust flow around the periphery of the exposure passage and into the exhaust passage before the aerosol was evacuated through an array of three ports located on the back plate of the exhaust passage. The total displacement volume of the inhalant exposure system was approximately 1.4 liters.

The total system flow rate was 10 L/min with 7.5 L/min supplied to the aerosol generator, and 2.5 L/min supplied as dilution air resulting in a flow velocity of approximately 0.3 meters per second. At the tested flow rate, the total system air changes were approximately seven per minute. A Collison 3-jet nebulizer (BGI Inc., Waltham, Mass.) was used to aerosolize the biological agent, B. anthracis (Ames strain), and the biological agent simulant Bacillus globigii (hereafter B. globigii) for testing. Filtered house air was provided to supply a continuous and regulated air source to the Collison nebulizer and for additional dilution air. The Collison nebulizer flow rate was maintained at approximately 7.5 L/min by supplying a continuous and regulated air supply to the Collison at 30 psi, and the flow rate was monitored using a Sierra 0 to 20 L/min mass flow meter (Sierra Instruments, Monterey, Calif.). Dilution airflow was controlled with a needle valve at 2.5 L/min and was monitored using a Sierra 0 to 10 L/min mass flow meter. The Collison nebulizer by-pass airflow was controlled using a needle valve at approximately 7.5 L/min and was monitored using a Sierra 0 to 20 L/min mass flow meter. The bypass flow was used to maintain system pressure and flow stability when the Collison nebulizer was not in use. During testing, the system was maintained under a slight negative pressure of approximately 0.127 cm (0.05 inch) of H₂O to avoid contamination of the biological safety cabinet.

A test matrix was developed to characterize the exposure system performance related to inhalant properties such as aerosol concentration stability, aerosol size distribution, aerosol sampler evaluation, and test to test reproducibility.

System concentration stability tests were conducted using fresh 5 mL aliquots from the same B. globigii spore stock for each test. The B. globigii spore stock concentration was 8.08×10⁸ colony forming units per milliliter (cfu/mL) as measured by the spread plate technique and size distribution were measured using a model 3321 Aerodynamic Particle Sizer® Spectrometer (particle sizer) from TSI incorporated (St. Paul, Minn.). The analyzer was designed to accurately measure count and size distribution of particles with aerodynamic diameters in the range of 0.5 to 20 μm.

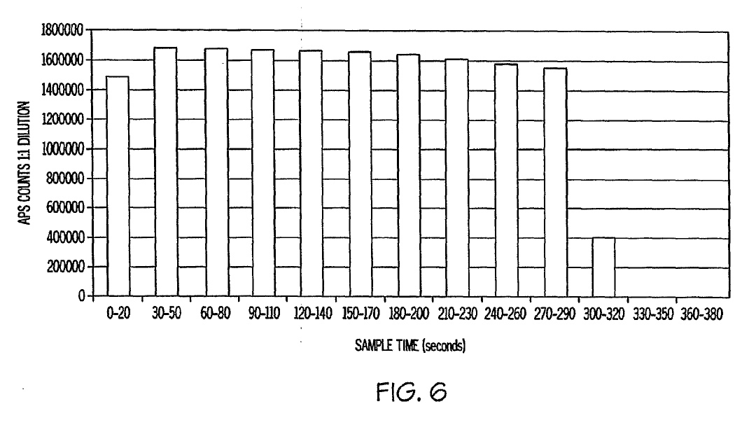

For stability testing, particle sizer samples were taken during the entirety of each test, and included measuring the post generation concentration decline until the system was purged of aerosol. FIG. 6 shows a representative graph of the R&D exposure system concentration profile relating particle counts verses time. Table 1 shows the count rates, Mass Median Aerodynamic Diameter (MMAD), Geometric Standard Deviation (GSD), and Standard Deviation of the MMAD.

TABLE 1
Summary of Raw Data of B. globigii Aerosol Counts

| Time (sec) | Counts | MMAD | GSD | MMAD (SD) |
|---|---|---|---|---|
| 0-20 | 1488768 | 1.01 | 1.48 | 0.01 |
| 30-50 | 1681565 | 1.00 | 1.52 | |
| 60-80 | 1673562 | 0.98 | 1.49 | |
| 90-110 | 1665980 | 0.98 | 1.49 | |
| 120-140 | 1660286 | 0.98 | 1.50 | |
| 150-170 | 1658746 | 0.99 | 1.51 | |
| 180-200 | 1642684 | 0.99 | 1.51 | |
| 210-230 | 1609523 | 0.99 | 1.50 | |
| 240-260 | 1581197 | 0.98 | 1.50 | |
| 270-290 | 1554731 | 0.98 | 1.51 | |
| 300-320 | 405119 | 0.99 | 1.50 | |
| 330-350 | 491 | 0.98 | 1.49 | |
| 360-380 | 186 | 0.97 | 1.50 | |

### Current Aerosol System Stability Testing

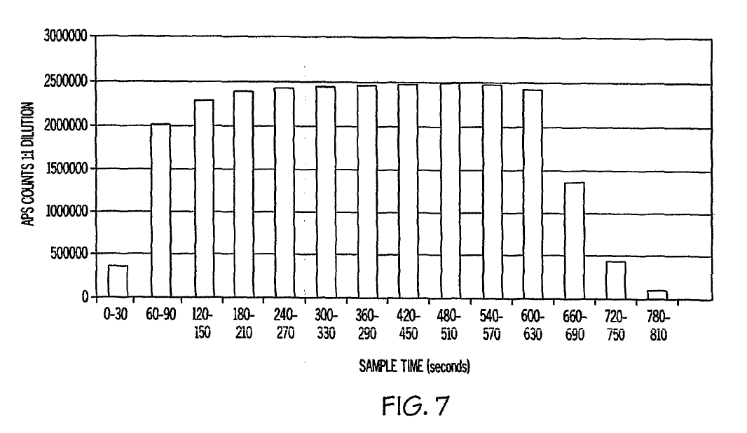

Referring now to FIG. 7, the figure shows a representative graph of the exposure system concentration stability profile of the exposure system that was used. The concentration profile relates particle counts verses time. System concentration stability tests were conducted using a fresh 8 mL aliquot from the same B. globigii spore stock. The B. globigii spore stock concentration was 1.09×10⁹ colony forming units per milliliter (cfu/mL) as measured by the spread plate technique.

The particle size analyzer was programmed to pull sequential samples from the exposure system for 30 seconds starting at the initiation of aerosol generation with a 30 second delay between samples. A total of fourteen particle sizer samples were collected. This included measuring the post generation concentration decline for four minutes after the 10-minute aerosol generation period. The flow rate through the Collison nebulizer was maintained at 7.5 L/min with a dilution airflow rate of 8.5 L/min for a total system flow rate of 16.0 L/min. These flow rates were used to simulate system operation parameters used during actual exposure testing.

Testing showed that the current aerosol system maintains a peak aerosol concentration after about a 3 to about 4 minute ramp-up time.

### Aerosol System Particle Size Testing

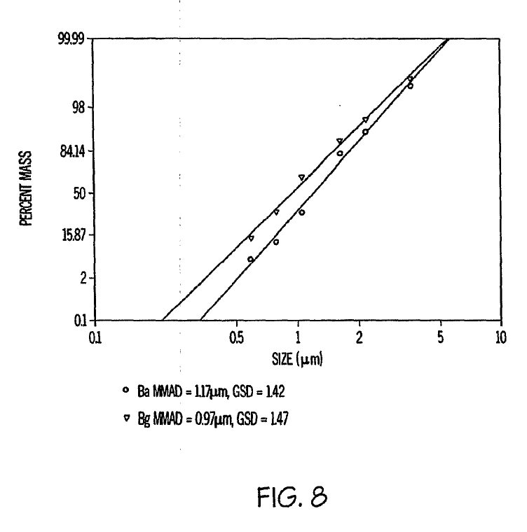

Twelve individual 5 minute inhalation exposure system tests were conducted using a fresh 5 mL aliquot from the same B. globigii spore stock with a concentration of 8.08×10⁸ colony forming units per milliliter (cfu/mL). Nine 10 minute tests were also conducted using a fresh 5 mL aliquot from the same B. anthracis spore stock (Lot number Ames-B8) with a concentration of 1.0×10⁷ colony forming units per milliliter (cfu/mL). Particle sizer samples were taken for a duration of 30 seconds at the midpoint of each B. globigii and B. anthracis test to compare test to test stability and/or variation of the particle size distribution. FIG. 8 shows a log-probability plot representing the average of all particle size distributions obtained for all B. globigii and B. anthracis tests. The mass median aerodynamic diameter (MMAD), geometric standard deviation (GSD), and MMAD standard deviation are also shown.

Data obtained from testing the inhalant exposure system shows promising results with applications for bioaerosol studies in the primate and rabbit models. The aerosol concentration stability test results from FIG. 7 and Table 1 show a very short time lapse for concentration stable state stability and equilibrium, approximately 15 to 30 seconds and maintains stable peak aerosol concentration for the duration of the aerosol exposure. The aerosol concentration decay after the aerosol generator (Collison nebulizer) is turned off also shows a very short aerosol purge time lapse, approximately 30 to 60 seconds. This short time duration aerosol concentration stability and decay are advantages for accurate and reproducible aerosol exposures and measurement. Stability testing showed reproducible and very nominal variation comparing test to test counts and the B. anthracis test concentration profile over time also showed a similar trend when compared to the B. globigii tests.

The inhalation exposure system has shown superiority by having a low displacement volume, a rapid development to peak aerosol concentration, a stable peak aerosol concentration, a rapid decay of agent, sampling directly from the aerosol stream for accurate aerosol concentration determination, decreased aerosol residence time, and the potential for decreasing the aerosol exposure duration that conserves biological agent.

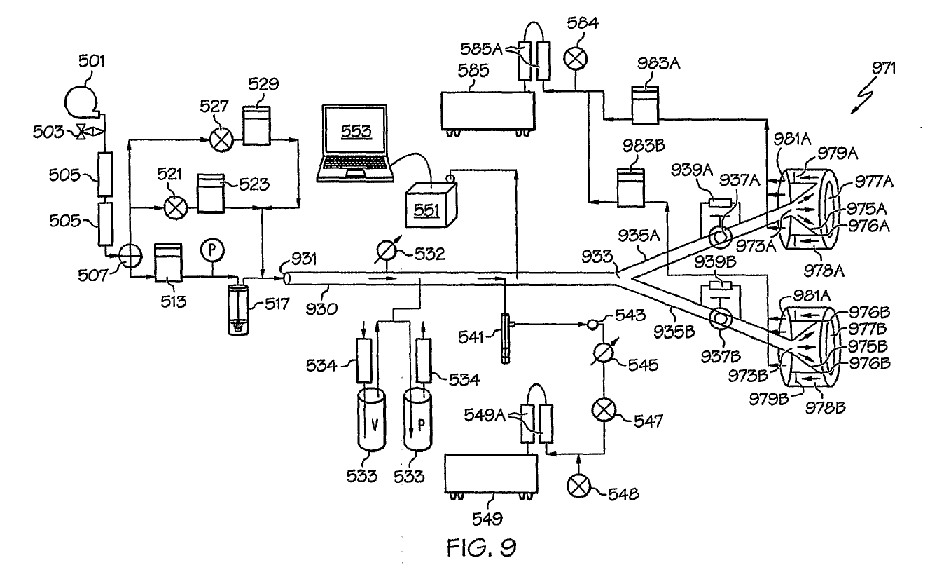

Referring now to FIG. 9, this figure illustrates a typical dual inhalant exposure unit for two animals with optional sensing instruments. Where components are the same as in FIG. 5, the numbering of FIG. 5 has been retained for simplicity. A flow of air from a source 501 is controlled by pressure regulator 503 before flowing to one or more filters 505 (e.g. HEPA filters). A three way valve controls flow directly to a nebulizer 517 via a mass flow controller 513 and pressure gauge 515. A flow of dilution air flows via a pressure regulator 521 to mass flow controller 523 directly to the inlet 531 of tube 530. An additional flow path for bypass air involves flow from valve 507 to pressure regulator 527 and mass flow meter 529 and then directly to the tube inlet 531. Aerosol produced in the nebulizer 517 flows directly to the inlet 931 of tube 930.

The length of tube 930 is any that provides a good flow aerosol flow path, distribution to sensing instruments and proper delivery to the inlet 933 of dual tubes 935A, 935B that provide flow to inlets 973A, 973B of the dual inhalant exposure unit 971. A differential pressure gauge 532 is typically used to monitor pressure in tube 930. Vacuum and pressure relief vessels 533 along with associated filters 534 (e.g. HEPA filters) may be used to control pressure in the tube 930. A sample collector such as an impinger 541 may connected to the tube 930 to collect aerosol particles. A critical orifice 543, along with vacuum gauge 545, valve 548 and a vacuum pump 549 along with filters 549A may be used to aid in collecting the samples. In one embodiment the critical orifice provides a flow of air of 2 L/min. An optional aerodynamic particle sizer 552 connected to the tube 530 may be used with a computer 553 to aid in monitoring and controlling particle size.

Aerosol and air then flows to the inlet 933 two tubes 935A, 935B of the dual portion of inlet 573 of the inhalant exposure unit 571 from which the flow is directed to a cones 595A, 975B where an animal's mouth and nose are typically placed via ports 977A, 977B. The unused aerosol and air along with exhaled air from the animal flows out of the cone 975A, 975B into a concentric inlet 576A, 975B to a typically concentric exhaust passage 978A, 978B. Exhaust passage 978A, 978B contains a flow restrictor 979A, 979B that controls flow out of the exhaust passage and provides for increased air flow where the animal breathes in the cone 975A, 975B. This is accomplished by a vacuum applied at an outlet port 981A, 981B of the exhaust passage 978A, 978B. Flow restrictor 979A, 979B essentially controls the effects of this applied vacuum in the cone 975A, 975B. As mentioned earlier the effect of the vacuum and flow restrictor 979A, 979B is to is increase the speed of air flow at the animal's nose or mouth above the air flow provided by the inflow of air and aerosol to the cone. This has the effect of reducing rebreathing of exhaled air by the animal. Exhaust air flows from outlet port 981A, 981B to a mass flow controller 983A, 983B then to an optional bypass valve 584, or to an exhaust pump 585 (with filters 585A) that provides vacuum at the outlet ports 581.

Additionally, the dual tubes 935A, 935B have control valve 937A, 937B that controls the flow of air and aerosol to the animal. A bypass filter 939A, 939B is used to supply air flow to an animal without aerosol when the valve 937A, 937B is turned off. These valves are also referred to as isolation valves.

### Example 2

### Multiple Animal Inhalation Exposure System

The multiple inhalation exposure system (FIG. 9 ) was constructed of Plexiglas, and consisted of a 2.54 cm inside diameter tube with a 5.08 cm outside diameter of approximately 56 cm long. The end of the tube was mated with a Y tubing connector with an inside diameter of 1.9 cm. The Y tubing connector is utilized to divert the challenge aerosol to two separate exposure sites. Two 50 cm long sections of flexible Tygon™ tubing with a 1.9 cm inside diameter are connected to each port of the Y connector. The two sections of Tygon™ tubing were in turn connected to two ball valves (also known as isolation valves) that are each attached to an exposure unit. The ball valves would be utilized in actual exposure challenges to turn off the exposure challenge to one of the exposure units and animal model based on inhaled volume while continuing to deliver the exposure challenge to the other exposure unit. By tuning the ball valve off, the aerosol challenge is redirected around the ball valve and HEPA filtered before reentering the exposure unit downstream of the ball valve thus supplying fresh air to the animal during post exposure washout of the system. The two inhalation exposure units consisted of a 10.2 cm long and 5.1 cm inside diameter solid stock of Plexiglas with a 10.2 cm outside diameter. The end of each unit was lathed at 45° to form a cone radiating out from the 2.54 cm (1 inch) diameter inner tube, to the 10.2 cm (4 inch) diameter outer tube for the insertion of the animals nose through a rubber dam. A 15.25 cm (6 inch) outside diameter and 12.7 cm (5 inch) inside diameter Plexiglas™ tube was mounted concentrically with front and back plates around the 4 inch diameter tube for exhausting the aerosol from each exposure unit. The exhaust passage and face plate, located around the animal nose insertion region, encompasses the cone and is spaced with a gap of about 3 mm. The small gap increases the acceleration of resident aerosol that will pass the animal's nose to facilitate the replenishment of fresh—low residence time biological aerosol in the animal's respiration zone to prevent rebreathing of exhaled air. The aerosol then enters the exhaust passage which contains a flow restrictor which is separated about 4 mm from the exhaust outer housing. The flow restrictor acts as an exhaust flow distributor to maintain a consistent exhaust flow around the periphery of the exposure tube and into the exhaust passage before the aerosol is evacuated through an array of three outlet ports located on the back plate of the exhaust passage. The total displacement volume of the exposure system is approximately 1.1 liters; excluding the exhaust volume of the exposure units exhaust passage.

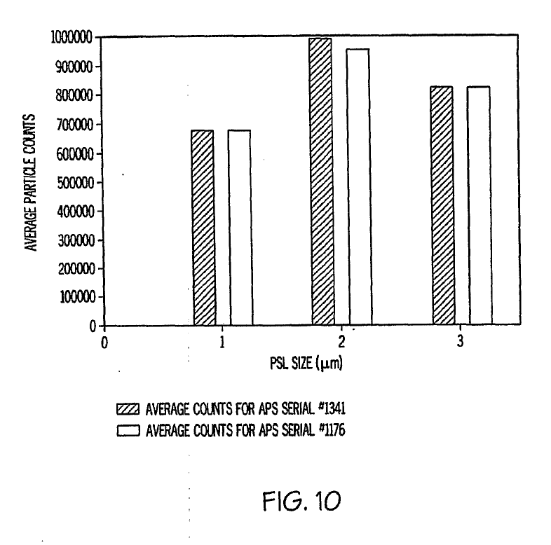

Referring now to FIG. 10, this figure is a bar graph of a particle sizer correlation. The horizontal scale shows polystyrene latex microsphere size (um) and the vertical scale shows the average particle counts. Bar set 1 shows average counts for two tests with the 0.993 um particles, Bar set 2 shows average counts for two tests with the 1.992 um particles, and Bar set 3 shows average counts for two tests with the 2.92 um particle sizes. The correlations are very good and are calculated at about 1% for set 1, 3% for set 2 and 0.5% for set 3.

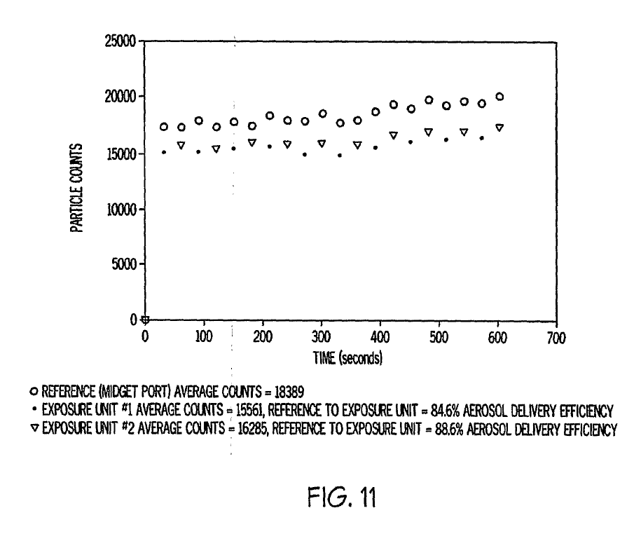

Referring now to FIG. 11, this figure shows a graph depicting PSL system homogeneity and aerosol delivery efficiency results for 0.993 um particles. The horizontal scale is Time in seconds and the vertical scale is the number of particle counts. The open circles (upper curve) are reference counts (average of 18,400). The black dots (lower curve) are counts for Exposure unit #1 (average counts 15,600). Exposure unit #1 had about an 85% aerosol exposure delivery efficiency. The triangles depict data for Exposure Unit #2 (average counts 16,300). Exposure unit #2 had about a 89% aerosol delivery efficiency. The two units had about a 96% count percent correlation.

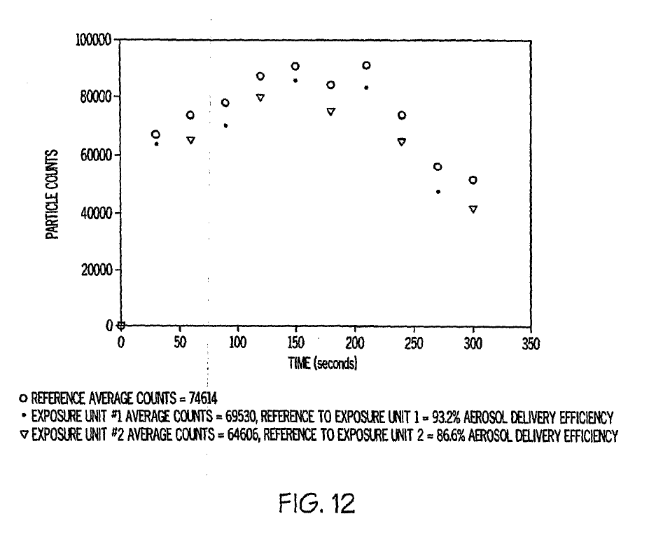

Referring now to FIG. 12, this figure is graph depicting PSL system homogeneity and aerosol delivery efficiency results for 1.992 um particles. The horizontal scale is Time in seconds and the vertical scale is the number of particle counts. The open circles (upper curve) are reference counts (average of 74,600). The black dots are counts for Exposure unit #1 (average counts 69,500). Exposure unit #1 had about a 93% aerosol exposure delivery efficiency. The triangles depict data for Exposure Unit #2 (average counts 64,600). Exposure unit #2 had about a 87% aerosol delivery efficiency. The two units had about a 93% count percent correlation.

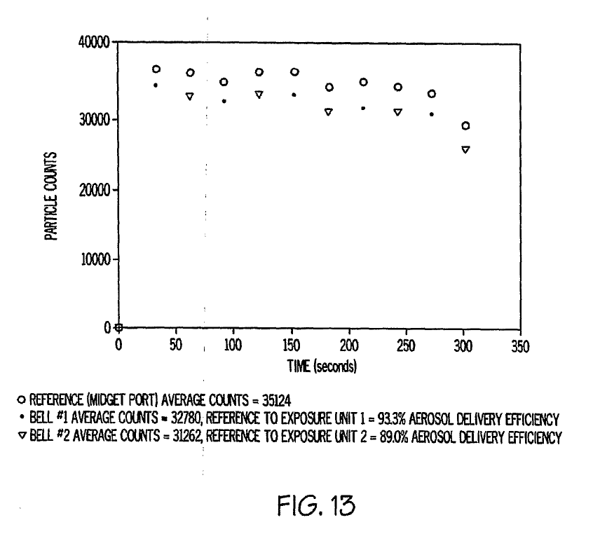

Referring now to FIG. 13, this figure shows a graph depicting PSL system homogeneity and aerosol delivery efficiency results for 2.92 um particles. The horizontal scale is Time in seconds and the vertical scale is the number of particle counts. The open circles (upper curve) are reference counts (average of 35,100). The black dots are counts for Exposure unit #1 (average counts 32,800). Exposure unit #1 had about a 93% aerosol exposure delivery efficiency. The triangles depict data for Exposure Unit #2 (average counts 31,300). Exposure unit #2 had about a 89% aerosol delivery efficiency. The two units had about a 96% count percent correlation.

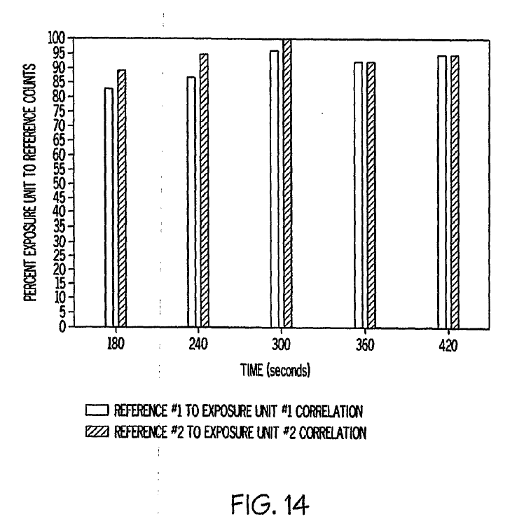

Referring now to FIG. 14, this figure shows a bar graph of B. anthracis aerosol delivery efficiency. The horizontal scale is Time in seconds. The vertical scale is in % and plots the percent exposure unit to reference counts. The graph shows the reference (midget port) to exposure unit aerosol delivery efficiency.

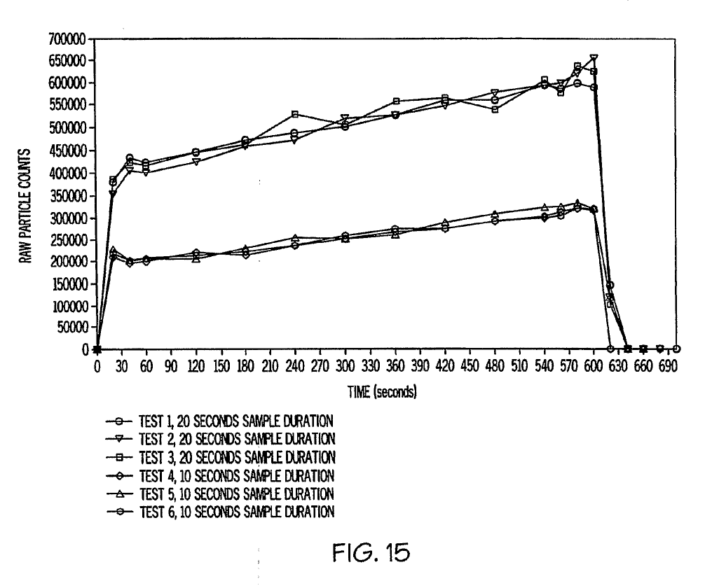

Referring now to FIG. 15, this figure shows a graph of B. anthracis aerosol stability. The horizontal scale shows Time in seconds and the vertical scale shows Raw Particle Counts. Tests 1, 2 and 3 were of 20 seconds duration. Tests 4, 5, and 6 were of 10 second duration. The curves show a quick rise time and an essentially flat particle count over the measurement period. The curves rise somewhat due to an increase in the generator (Collision nebulizer) suspension particle concentration over time related to a preferentially higher dissemination rate of the carrier liquid than particles over the test period.

### Example 3

Aerosol Challenge (Nebulizer) Suspension Enumeration: The challenge spore suspensions (B. anthracis) were prepared by diluting the stock suspension to a targeted concentration. The challenge spore suspension was enumerated by serial dilution of the challenge suspension by spreading 0.1 mL on each of five tryptic soy agar plates for three different dilutions. The tryptic soy agar plates were placed in a secondary container and incubated at 37° C. for 16-24 hours. After the incubation period, the number of colonies on each plate was counted. Each concentration was determined by the spread plate method.

### Example 4

As tested, the total system flow rate for all testing was 20 L/min, resulting in a flow velocity of approximately 0.66 meters per second through the main delivery tube, and a velocity of 0.51 meters per second through each section of the Tygon aerosol delivery tubing. A total of 2.5 liters of the total flow is sampled from the main aerosol delivery tube before the aerosol is diverted to the two Tygon tubes and delivered to the exposure units. The diverted air flow supplied to each exposure unit is maintained at a flow rate of approximately 8.75 L/min using mass flow controllers (Sierra Instruments, Monterey, Calif.) for control of the exhaust flow of each exposure unit. At the tested flow rate, the total system air changes are approximately sixteen per minute.

### Example 5

Simulant Testing: The objective of this testing was to characterize the exposure system and assess individual parameters of the exposure system which include aerosol homogeneity, concentration ramp up, concentration stability, and decline, as well as aerosol transport losses, sample measurement to exposure location concentration variation, exposure location to exposure location variation, and sampling system collection efficiencies. To characterize these system parameters, individual polystyrene latex microsphere standards were prepared at sizes of 0.993, 1.992, and 2.92 um suspended in solutions of deionized sterile water and reagent grade ethanol. To accurately characterize and assess the exposure system, two particle sizers TSI inc. St. Paul, Minn. were used in tandem sampling simultaneously at separate locations in the exposure system for comparative count concentration measurements. The particle sizer's were concentration count rate correlated with each polystyrene latex suspension size prior to all characterization testing. This was performed to measure the count concentration measurement variation between the two instruments and to correct for concentration count and mass concentration measurement results obtained from characterization testing. The particle sizers were correlated by aerosolizing each individual size suspension into a small plenum using a Westmed Vixone™ disposable nebulizer, and sampling simultaneously with both instruments from the same location at the same sample flow rate from the plenum. FIGS. 11-13 show a graph with the count concentration correlation results for both instruments for inhalant exposure systems and for each polystyrene latex microsphere size.

### Example 6

### Exposure System Homogeneity Testing:

For exposure system homogeneity characterization tests, one particle sizer was utilized to sample from the impinger sample location (reference) and the other particle sizer was used to alternately sample from both exposure locations. The particle sizer's were synchronized to sample simultaneously from both locations to measure the variation in aerosol count and mass concentration for each polystyrene latex microsphere size. To generate the challenge aerosol for each polystyrene latex microsphere size, an individual Vixone nebulizer was used for each suspension size to avoid suspension cross contamination. The Vixone nebulizers were operated in the range of 5 L/min with additional aerosol dilution air supplied to the system to obtain a total flow of 20 L/min. During testing, the system was maintained under a slight negative pressure of approximately 0.127 cm (0.05 inch) of H₂O to avoid contamination of the test environment.

FIGS. 11, 12, and 13 show graphs of the results obtained from exposure system count concentration homogeneity testing for 0.993, 1.992, and 2.92 um diameter polystyrene latex microspheres. The results are calculated from averaging multiple particle sizer count measurement results obtained from each location over a period of 10 minutes and calculating the percent count correlation from sample location to sample location. These results also represent system related aerosol count and mass transport losses from the reference location to each exposure location.

### Example 7

### Bioaerosol Testing:

A modified Microbiological Research Establishment type three-jet Collison nebulizer (BGI, Waltham, Mass.) with a precious fluid jar was used to aerosolize the biological agent, B. anthracis (Ames strain) from a water suspension. B. anthracis spores with a stock concentration of 6.5×10⁸ colony forming units per milliliter (cfu/mL) as measured by the spread plate technique.

Air was supplied to the aerosol system by an in-house system filtered through a high efficiency particulate (HEPA) capsule filter. A Collison 3-jet nebulizer (BGI Inc., Waltham, Mass) was used to aerosolize the biological agent, B. anthracis (Ames strain). Filtered house air was provided to supply a continuous and regulated air source to the Collison nebulizer and for additional dilution air. The Collison nebulizer flow rate was maintained at approximately 7.5 L/min by supplying a continuous and regulated air supply to the Collison at 27 psi, and the flow rate was monitored using a Sierra 0 to 20 L/min mass flow meter (Sierra Instruments, Monterey, Calif.). Dilution airflow was controlled with a needle valve at 12.5 L/min and was monitored using a Sierra 0 to 20 L/min mass flow meter. The Collison nebulizer by-pass airflow was maintained at approximately 7.5 L/min and was controlled using a Sierra 0 to 20 L/min mass flow controller. The bypass flow was used to maintain system pressure and flow stability when the Collison nebulizer was not in use. Air flow delivered to each exposure unit was maintained at a flow rate of approximately 8.75 L/min using mass flow controllers (Sierra Instruments, Monterey, Calif.) for control of the exhaust flow of each exposure unit. During testing, the system was maintained under a slight negative pressure of approximately 0.127 cm (0.05 inch) of H₂O to avoid contamination of the biological safety cabinet.

FIG. 14 shows system B. anthracis aerosol delivery efficiency results from reference (impinger sample location) to each exposure unit. Samples were taken alternately during B. anthracis aerosol generation using a single particle sizer sampling for 30 seconds from each location.

### Example 8

### System Stability

System concentration stability tests were conducted with B. anthracis spores with a stock concentration of 6.5×10⁸ colony forming units per milliliter (cfu/mL) as measured by the spread plate technique. Particle counts and size distribution were measured using a model 3321 Aerodynamic Particle Sizer® Spectrometer (particle sizer) from TSI incorporated (St. Paul, Minn.). The analyzer is designed to accurately measure count and size distribution of particles with aerodynamic diameters in the range of 0.5 to 20 μm. The particle sizer analyzer was programmed to pull sequential samples from the exposure system with no time delay between samples. This sequenced sampling was performed to measure the count rates at specific time intervals to determine when exposure system concentration stability is achieved. For stability testing, particle sizer samples were taken during the entirety of each test, and included measuring the post generation concentration decline until the system was purged of aerosol. FIG. 8 shows a representative graph of the R&D exposure system concentration profile relating particle counts verses time over a 10 minute period. Six individual tests were performed with the particle sizer taking 10 second sequential samples for three tests, and 20 second sequential samples for three tests as described in the graph legend. Due to the large quantity of data points acquired from particle sizer measurement for these tests, the points plotted on the graph are count measurements representing 60 second sample intervals from the one minute to nine minute time range.

### Example 9

### Sampler Testing

Midget Impingers model 7531-25 (Ace Glass Incorporated, Vineland, N.J.). For each test, three midget impinger were filled with 10 mL of sterile water from Sigma (St. Louis Mo.). The samplers were used to collect a representative fraction of the challenge aerosol from the midget impinger sample location as well as from exposure units 1, and 2. The impingers were operating simultaneously during each B. anthracis challenge test to measure variation in colony forming unit (cfu) concentration from location to location.

Five ten-minute tests were performed to evaluate system bioaerosol concentration variation. The samples were pulled from the exposure system during the entirety of an aerosol challenge test. The B. anthracis spore concentration collected by the samplers was measured by the spread plate technique.

For each test, the Collison nebulizer was filled with a fresh 8 mL aliquot of the B. anthracis stock suspension. The flow rate through the Midget impingers (3) sampling from the impinger sample port as well as exposure unit one and two, were each controlled at a flow rate of 2 L/min with a flow calibrated critical orifice from Lenox Laser (Glen Arm, Md.), by maintaining a negative pressure of 45.72 cm (18 inch) of Hg using a ⅕ hp vacuum pump (Gast Manufacturing, Benton Harbor, Mich.). Table 2 shows the sampler cfu collection data obtained for each test and exposure unit to exposure unit percent difference in cfu concentration.

TABLE 2
Dual Exposure System
Midget Impinger Results cfu/mL

| | | | | Exposure |
|---|---|---|---|---|
| | | | | Unit 1 & 2 |
| | A | B | C | Concentration |
| Test | Reference | Exposure | Exposure | Difference |
| Number | Location | Unit 1 | Unit 2 | (%) |
| 1 | 3.08 × 10⁵ | 4.32 × 10⁵ | 3.88 × 10⁵ | 10 |
| 2 | 2.06 × 10⁵ | 8.36 × 10⁵ | 11.50 × 10⁵ | 38 |
| 3 | 3.82 × 10⁵ | 6.08 × 10⁵ | 4.46 × 10⁵ | 27 |
| 4 | 3.00 × 10⁵ | 4.04 × 10⁵ | 5.36 × 10⁵ | 33 |
| 5 | 3.04 × 10⁵ | 4.90 × 10⁵ | 5.44 × 10⁵ | 11 |

Data obtained from testing the new inhalant exposure system showed promising results with applications for bioaerosol studies in the primate and rabbit models. The aerosol homogeneity test results from FIGS. 11, 12, and 13 showed a very nominal change in concentration from exposure unit to exposure unit, and also a very high aerosol delivery efficiency with the count concentration transport from the reference to exposure locations in the range of 80 to 95 percent for all polystyrene latex microsphere sizes. The results obtained from midget impinger collection efficiency tests (FIG. 7) also shows collection efficiency for each size range of polystyrene latex microsphere with particle collection in the range of 90 to 99 percent for single and duel impinger configurations at the 2 L/minute sample flow rate.

Bioaerosol aerosol delivery efficiencies from data in FIG. 14 also show high reference to exposure unit aerosol delivery efficiencies in the range of 83 to 98 percent, as well as very nominal differences in exposure unit to exposure unit aerosol concentrations.

The B. anthracis bioaerosol stability data FIG. 15 shows a short time lapse of approximately 15 to 30 seconds for the aerosol concentration to reach approximately 70% of the maximum concentration in each 10 minute test, and shows a very linear concentration dine up to the maximum concentration. The aerosol concentration decay after the aerosol generator (Collison nebulizer) is turned off also shows a very short aerosol purge time lapse of approximately 20 to 40 seconds for complete aerosol concentration purge. This short time duration aerosol concentration stability and decay are advantages for accurate and reproducible aerosol exposures and measurement. The results obtained from the bioaerosol testing in Table 1 show very reproducible results and a nominal difference in exposure unit to exposure unit aerosol cfu concentration based on the impinger enumeration results. The discrepancy in reference to exposure location results; although slight, with the reference cfu enumeration results being lower than the exposure location enumeration results is not consistent with results obtained from polystyrene latex microsphere delivery efficiency results which show the opposite effect. This phenomenon will need to be addressed with further characterization. Possible effects could include the sample velocity, sample probe geometry, and possibly a concentration gradient or non uniform aerosol concentration distribution in the delivery system at the impinger sample location. These results are significantly important for the collection and accurate quantification of respirable viable organisms delivered to the animal model for toxicity determinations.

The new aerosol system has shown superiority over a currently used aerosol system by having a lower displacement volume, a rapid development to peak aerosol concentration, a stable peak aerosol concentration, a rapid decay of agent, sampling directly from the aerosol stream for accurate aerosol concentration determination, decreased aerosol residence time, and the potential for decreasing the aerosol exposure duration. The ability to expose two or more animal models of the same or different species, and the use of a single sampler for the quantification of cfu's delivered to the animals will also conserve biological agent and personnel hours.

### Example For Flow Rate Calculation

Referring now to FIGS. 4 and 9, the system provides for advantageous flow rates whereby the flow past an animals face or nose is accelerated and rebreathing is minimized or avoided. This is accomplished by the proper sizing of openings D₁ and D₂. The following section discusses flow rate parameters that relate to this.

Flow=Q.

Exposure system flow Q at the main tube=20 L/min, Impinger Q=2.0 L/min, APS Q=0.5 L/min.

Exhaust flow total to animal Q=17.5 L/min÷2=8.75 L/min

Impinger and APS flows were removed from the calculation since the flow of gas to these units does not go to the animal; division is by two when there are two animals exposed simultaneously.

The exposure system gas flow Q to each animal is 8.75 L/min

Q=(8.75 L/min×0.001 m³/L)÷60 sec/min=1.46×10⁻⁴ m³/sec

Area of tube=n (pi) (0.0254 m²)÷4=5.06×10⁻⁴ m²

since Vel=Q/Area (1.46×10⁻⁴ m³/sec)÷5.06×10⁻⁴ m²

Vel=0.29 m/sec at the animals nose or mouth

This is the velocity of gas flow past the animal's nose or mouth and indicates that adequate flow is being provided to an animal such as a mouse or similar small animal to prevent rebreathing.

The aperture at D₂ provides for increased exhaust velocity at the point where the gas has passed the animal's nose or mouth. This is accomplished by having a negative pressure applied at the exhaust 477 and appropriate sizing of aperture D₂. D₁ is assumed to be large for this and can be ignored. However in some embodiments the aperture D₁ may be acting as a flow accelerator by itself if there is not aperture D₂ further down the flow path.

Calculation for aperture D₂ exhaust velocity

Aperture width—1.3 mm×2=2.6 mm

Area of aperture=n r² outer−n r²inner

Outer diameter=4 inches×25.4 mm/inch=1.01.6 mm=0.1016 m thus r=0.0508 m

Inner diameter=0.1016 m−0.0026 m=0.099 m thus r=0.0495 m

Area of aperture D₂=n (0.0508 m)²−n (0.0495)²=0.0004 m²

Thus the gas flow velocity at the aperture is

V=Q/A=1.46×10⁻⁴ m³/sec÷4.0×10⁻⁴ m²=0.365 m/sec

Thus the gas flow rate is accelerated by the aperture.

If D₁ is the flow constrictor than it would be used in the calculation, however the preferred flow constrictor is at D₂.

While the forms of the invention herein disclosed constitute presently preferred embodiments, many others are possible. It is not intended herein to mention all of the possible equivalent forms or ramifications of the invention. It is to be understood that the terms used herein are merely descriptive, rather than limiting, and that various changes may be made without departing from the spirit of the scope of the invention.

We claim:

1. An inhalant exposure unit comprising:
a. a housing concentrically positioned around a central axis having an inlet and an outlet;
b. a face plate positioned vertically to the central axis at the outlet of the housing, wherein the outlet of the housing and a surface of the face plate are separated by a distance D₁ comprising an annular outlet between the outlet of the housing and the surface of the face plate; and
d. an opening in the face plate for admitting at least a portion of an animal's head into the housing.

2. The inhalant exposure unit according to claim 1, wherein at least a portion of the housing comprises a truncated cone having a surface at an angle θ with respect to the central axis, wherein the smaller end of the cone comprises an inlet, and the outlet is at the larger end of the cone.

3. The inhalant exposure unit according to claim 1 or 2, comprising an outer housing concentrically located around the housing wherein the outer housing and the housing form an exhaust passage connected to the annular outlet for exhausting inhalant and an animal's exhaled breath.

4. The inhalant exposure unit according to claim 3, wherein a flow restrictor is located in the exhaust passage.

5. The inhalant exposure unit according to claim 3, wherein the flow restrictor is located concentrically within the exhaust passage and forms an annular exhaust orifice.

6. The inhalant exposure unit according to claim 3, wherein the exhaust orifice has an annular outlet of distance D₂.

7. The inhalant exposure unit according to claim 3, wherein the flow restrictor is located a distance D₅ from the exhaust.

8. The inhalant exposure unit according to claim 1, wherein the annular outlet comprises an annular gap without a support across the gap.

9. The inhalant exposure unit according to claim 1, wherein annular outlet comprises an annular gap with a least one spaced support across the gap.

10. The inhalant exposure unit according to claim 1, wherein the annular gap comprises a distance D₁.

11. The inhalant exposure unit according to claim 1, wherein an essentially flexible seal located concentrically with respect to the central axis contacts at least a portion of the face plate, and has a central orifice for admission of an animal's head or muzzle.

12. The inhalant exposure unit according to claim 3, wherein an outlet port at the exhaust passage comprises a plurality of holes.

13. The inhalant exposure unit according to claim 3, wherein the flow restrictor comprises an annular ring that blocks a portion or all of the exhaust passage, and has a plurality of holes.

14. The inhalant exposure unit according to claim 2, wherein the angle θ ranges from about 0° to about 50°.

15. The inhalant exposure unit according to claim 14, wherein the angle θ ranges from about 10° to about 40°.

16. The inhalant exposure unit according to claim 1 or 2, wherein the unit Has a unitary structure.

17. The inhalant exposure unit according to claim 2, wherein the outer housing, housing, an optional inlet tube, and truncated cone are essentially concentric about the central axis.

18. A method for testing an animal with an inhalant comprising;
a. providing an inhalant exposure unit according to claim 1;
b. placing an animal's head or muzzle within the opening of the face plate; and
c. flowing an inhalant into the inlet.

19. A multiple inhalant exposure system comprising:
a. two or more inhalant exposure units according to claim 1 or 2; and
b. a distributor having an inlet for inhalant and two or more distribution tubes, wherein each tube has an outlet operationally connected to the inlet of each inhalation exposure unit.

20. An inhalation exposure system comprising;
a. an inhalant generator;
b. a tube with an inlet and an outlet, wherein the inlet is connected to the output of the inhalant generator; and
c. an inhalation exposure unit according to claim 1 or 2, wherein the inlet of the inhalation exposure unit is connected to the tube outlet.

21. An inhalation exposure system for treatment of a patient comprising;
a. an inhalation generator for providing an aerosol or powder;
b. an inhalation exposure unit having an inlet connected to the inhalation generator comprising:
1. a tapered exposure chamber having a narrow and a wide end, with the inlet at the narrow end of the chamber and having a port at the wide end of the chamber that accommodates at least a part of a patient's head for breathing from the exposure chamber;
2. an exhaust passage, having an inlet connected to the wide portion of the tapered exposure chamber, and having an outlet;
3. a flow restrictor in the exhaust passage; and
c. a vacuum unit that provides a vacuum at the outlet of the exhaust passage.

22. The inhalation exposure system according to claim 21, wherein the inhalation generator is a nebulizer.

23. The inhalation exposure system according to claim 21, wherein the patient to be treated is a human or animal.

24. The inhalation exposure system according to claim 21, wherein the vacuum unit is a pump.

25. The inhalation exposure system according to claim 21, wherein the exhaust passage and its inlet is substantially concentric to the chamber.
